(19) United States
(12) Patent Application Publication
(10) Pub. No.: US 2011/0023789 A1
(43) Pub. Date: Feb. 3, 2011

(54) GAS DELIVERY SYSTEM FOR AN ANIMAL STORAGE CONTAINER

(75) Inventor: George Delagrammatikas, Whitestone, NY (US)

Correspondence Address: GOODWIN PROCTER LLP;ATTN: PATENT ADMINISTRATOR, 620 Eighth Avenue, NEW YORK, NY, 10018, US

(73) Assignee: THE COOPER UNION FOR THE ADVANCEMENT OF SCIENCE AND ART, New York, NY (US)

(21) Appl. No.: 12/735,021

(22) PCT Filed: Dec. 9, 2008
(86) PCT No.: PCT/US2008/086023

§ 371 Date: Oct. 15, 2010

Related U.S. Application Data
(60) Provisional application No. 61/007,188, filed on Dec. 10, 2007.

(51) Int. Cl. A61M 16/00

(52) U.S. Cl. 119/420

(57) ABSTRACT

This invention is directed to a system and method for delivering gases, such as air, oxygen, carbon dioxide, carbon monoxide, and cigarette smoke, to a laboratory animal storage container. The system and method of the present invention allow for tight control and monitoring over the immediate environment of the animal, and allow the environment to be varied rapidly during a study. The invention is particularly useful for sleep apnea studies, but has broad application to animal studies in general.

### CROSS REFERENCE TO RELATED APPLICATIONS

This application claims the benefit of priority of U.S. provisional application 61/007,188 filed on Dec. 10, 2007 which is incorporated by reference herein.

### BACKGROUND OF THE INVENTION

1. Field of the Invention

This invention is directed to a gas delivery system used for delivering gases such as, but not limited to, air, oxygen, carbon dioxide, carbon monoxide, and cigarette smoke to a laboratory animal storage container, and to a method of using such a gas delivery system.

2. Background Information

The need to rapidly vary and precisely control the proportions of different gases in laboratory animal storage containers is widespread in life science research, affecting many in vitro and in vivo applications. One such instance relates to sleep apnea, where numerous animal models exist that simulate the effects of chronic intermittent oxygen starvation on the human physiology.

Current state-of-the-art devices generally include containments (as used herein, containment refers to either an external container that houses one or more individual animal containers, or an individual animal container itself) integrated with gas delivery systems that cycle the oxygen levels between selected concentrations, within a prescribed time and an imprecise vicinity of the animals. Control is performed through traditional closed- and open-loop feedback algorithms based on predefined timing schedules, or even activated directly by the animal's brainwave signals and arousal state. One of the several drawbacks with currently available systems is that they do not perform as well in studies where the environment in the immediate vicinity of the animal must he changed rapidly and intermittently, in a controlled and precise fashion. In the present invention, this situation is rectified, in part, by providing improved circulation in the animal cage or container itself, by use of active mixing devices, such as a fan. None of the currently available systems provide such effective active mixing where it is most important, within the animal container or cage.

Sleep apnea is a condition characterized by the cessation of breathing when a person is asleep. This condition is divided into two general types, termed “obstructive” and “central,” which arise through fundamentally different mechanisms. The former usually occurs when a person sleeps on his or her back, whereby the tongue blocks the airway as it falls back against the soft palette. Increased fatty deposits around the throat region may also constrict the airway, thereby further preventing a person's adequate breathing. Studies have shown that the combination of air passage geometry, its deformability, and the negative pressure induced in the throat while breathing draws the airway closed. These three conditions, and any combinations thereof, are collectively termed “obstructive sleep apnea” (OSA).

The improper function of a person's respiratory control centers causes central sleep apnea (CSA). Unlike OSA, the effort to breathe is either diminished or nonexistent in CSA sufferers. They lose this drive to breathe through a variety of mechanisms that cannot always be decoupled from one another. These causes include: 1) decreased sensitivity of receptors to the chemical signals the body produces in order to initiate breathing; 2) hypoxia, which promotes CSA's severity, caused by altitude changes, as well as low blood-oxygen content while a person is sleeping; 3) obesity, suggesting a chemical component may promote CSA; and 4) sleep-state related effects, especially during the transition between sleep and wakefulness.

In either case of sleep apnea, the level of oxygen in the blood varies dramatically and rapidly throughout sleeping hours. General terms for oxygen concentration levels in the environment include normoxic (normal levels), hypoxic, and hyperoxic (lower and higher than normoxic, respectively).

Neurobiologists have been studying the effects of sleep apnea in both humans and laboratory animals for many years. Of specific interest is the study of hypoxia-induced physiological effects in laboratory rodents, such as hypertension, emotional states, cognition, metabolism, etc. To simulate the effects of sleep apnea in humans, researchers have proposed animal models in which the oxygen concentration in the laboratory animal cages and nearby vicinity can be cycled between normoxic (20.9% O₂) and hypoxic (˜5-10% O₂) conditions very rapidly. Animal models used to study sleep apnea typical vary from normoxic to hypoxic conditions (and vice versa) over durations of several seconds to several minutes.

The most common containment systems to create locally controlled environments for studying hypoxia-induced effects on rodents range from large containment chambers that can house multiple, commercially-available animal cages simultaneously, to purpose-built (i.e., custom-designed) animal cages or containers of varying geometries and aspect ratios. Both commercially available and purpose-built systems typically include integrated gas delivery, detection, and exchange capabilities, however, these systems lack the precision and control needed for sophisticated animal experiments—they do not provide for adequate detection, control, and homogeneity of environment in the immediate vicinity of the animal.

While one aspect of sleep apnea studies is to be able to control the oxygen levels within the chambers (both the containment chambers and animal cages), another important aspect is to measure the oxygen concentration within the laboratory animal's body. The oxygen concentration in a laboratory animal's body can be measured by different methods, which vary in accuracy, invasiveness, and expense. Determining the blood oxygen content (i.e., the oxyhemoglobin content) is one of the most common methods to assess the oxygen concentration in a laboratory animal's body. The ultimate objective of most sleep apnea studies is to correlate oxyhemoglobin content with the sleep apnea-related physiological effects of interest; in practice, this in turn depends upon how well the environmental conditions in an animal study can be controlled.

Much confusion has arisen in the literature regarding the link between oxygen levels in the environmental surroundings of the laboratory animal and the oxygen levels within that animal's blood. Some research suggests that a hypoxic environment of 10% oxygen yields oxyhemoglobin levels of about 70% (where ˜95% oxyhemoglobin suggests a normoxic environment). This hypoxemic condition, (lower than normal oxygen level in the animal's blood), is a very loose function, at best, of the environmental oxygen conditions as measured during a typical sleep apnea study. There are two main reasons for the loose correlation.

First, the oxygen detection device used in most studies is typically placed relatively far from the animal, with respect to the mixing length scales of the gases of interest inside the animal cage (i.e., gases supplied into the cages during a sleep apnea study in addition to the gases that exist in the environment and those that are produced by the animal). Mixing length scale is a general indication of how quickly and how well gases from different sections of a container mix and reach uniformity. Second, the ability of a gas supplied or delivered into the containment chambers to mix uniformly throughout the chamber and animal cages (i.e., the mixing potential) depends on several factors, including: 1) the geometries of the animal cage and the surrounding containment chambers; 2) the number of animal cages in the chamber; and 3) the orientation of each animal cage within the containment chamber.

Together, these factors contribute to difficulties in the ability to accurately control and measure the environmental oxygen (or other gases of interest) in an experimental system, and consequently, for example, to highly unpredictable oxyhemoglobin levels in the test animals for a given set of environmental conditions. Also, improved control and homogeneity would result in lower gas consumption so that a study can be carried out with less waste and lower floor space requirements for gas tank storage.

Another challenge in studying sleep apnea in animal model experiments is the ability to vary oxygen levels reproducibly and in a highly controllable fashion, with improved sensitivity or response time to the user-specified oxygen levels. This requires an atmospheric control system (either open- or closed-loop feedback) applied to an open thermodynamic system (i.e., one in which mass flows occur across the boundaries of the cage). Furthermore, it would be useful to be able to record and analyze study data over the course of an experiment (e.g., the environmental conditions that are being measured for control purposes, experimental observation made, statistical reduction, and reporting of results and analyses).

A variety of rodent environmental systems are used in industry and academia. For large scale studies, modules of multiple rodent cages can be connected to central gas supply and outlet ducts. Each individual cage can tap into the central ducts, which supply gases at prescribed atmospheric conditions (oxygen, humidity, temperature, etc.). Such large scale systems or facilities are generally used as resting stops for a large population of rodents before they are subsequently handled in-house or delivered to other research facilities. The modules of rodent cages or containers are kept in a facility with ambient room air for the purpose of keeping the cages free of ammonia off-gas and high levels of carbon dioxide (i.e., there is no additional containment chamber enclosing the animal cages). A drawback of this system is that while there is some control of the environmental conditions via the supply and return ducts that connect with the modules, because of the system's large scale, the ability to vary conditions over the appropriate time scale for hypoxia studies is very challenging. Presently, hypoxia studies do not deal with the vast number of rodents involved in this type of design, which is why it is not implemented.

On the opposite end of the spectrum are smaller, individual animal cages, such as the types of cages used for the intensive care for post-operative care of animals. Such cages typically have a lid with variable area venting port(s) that can be manually set to open, closed, and intermediate positions. The venting ports allow the researcher to vary the gas exhaust rate in an open-loop manner, such that a predefined oxygen level is achieved. In addition, the supply gases may be delivered at the desired concentrations through a separate inlet port. Temperature can be controlled via a sensing device and a heating pad underneath the cage. A drawback of these small containers or cages is that they cannot deliver the level of homogeneity inside the cage without active gas mixing. Inhomogeneous gas pockets, or “dead zones” are present throughout these cages. These gas pockets mix passively due to bulk convective currents produced by rodent motion and the dissipation of the supply gas streams, as well as through thermal gradients within the cage. Another drawback is that because supply gases are typically provided or infused continuously, such systems may require substantial gas usage and storage space in the vicinity of the experiment.

Additional commercially available environmental chambers for controlling gas exchange include systems where the animal cage, having its own lid, is kept within a larger external chamber. The external chamber may have an opening so that gas supply and oxygen detection devices can be incorporated within the external chamber. A related design is where the external chamber comprises a plurality of shelves—the type of chamber used for environmental gas studies on cell cultures, where the cells are contained in open Petri dishes within the chamber. The gas exchange process affecting the cell cultures in these open dishes is rapid—the mixing length scales are also much smaller compared to the specimens and their locations. However, the same experimental control is not achieved in the macroscale applications of animal studies in rodent cages in similar chambers. Furthermore, animal studies using these systems typical require excessively large amounts of gas.

For convenience and cost savings, many researchers opt to build their own systems, having an outer containment chamber surrounding one or more standard rodent cages. The outer containment chamber may have a supply gas port, through which the supply gases can be provided from gas tanks to the outer chamber, and additional ports (in the external chamber and internal animal cages) through which a gas detection device can monitor the environment of the internal animal cage(s). Some designs even contain a circulation fan attached to the outer containment chamber to facilitate circulation of the environment within the outer chamber but external to the internal animal cage(s). These fans are typically undersized to have any meaningful mixing effect in the internal animal containers. Therefore, despite the inclusion of an outer containment chamber fan and gas monitoring devices, such systems still suffer from the drawback that they are not suitable for studies requiring the rapid and intermittent changes in environmental conditions of the internal animal cages. Such systems are better suited for long-term, chronic effect studies, where the location of the supply gas inlet stream, the volume of the large enclosure, and the placement of the gas detection device(s) are not critical to the control of the environment.

The animal cages discussed above resemble standard boxes composed of two main parts: the lower or bottom portion of the cage (i.e., the cage, container bottom, or container), and the lid, which allow the only pathways for gas exchange. Some designs include cages with openings in both the cage bottom (i.e., container) and the lid to enhance gas exchange. Such “flow-through” cages allow air to flow from a single inlet port located in the container, and leave through the top of the cage, through the lid. Together, the cage geometry, airflow rate, and intake and exhaust locations attempt to maximize the rate at which the internal air is replenished. Although such a design may help to enhance gas exchange and mixture homogeneity, the gas exchange is still passive, and suffers from the drawback of having slower response times when used in experiments where gas concentrations are designed to fluctuate. Flow-through cages are not widely used in the research setting.

The cages described above allow the animal to move freely within a confined space. These are not the only types of experimental conditions needed in scientific research. Animal cages are available that allow a rodent to be tethered to instrumentation. Such cages typically sit on a turntable, which rotates as necessary to prevent the tethering system from becoming entangled as the animal moves about the cage. A switch mechanism can be used to track the motion of the tether as the rodent moves, and send a signal to rotate the turntable to prevent tangling. These systems still suffer from lack of homogeneity and control in studies where the environment must be changed rapidly and intermittently.

From the discussion above, there is a need for improved environmental systems which can be used for animal studies that have the sensitivity and control required for sleep apnea and other animal studies. Accordingly, it is a first object of this invention to provide a gas delivery system and method for an animal storage container that translates to predictable and tightly controlled oxygen concentrations within the vicinity of the animal's body. The advantage of having more predictable control over oxygen concentration is that better conclusions can be drawn from the studies.

A second object of this invention is to provide a gas delivery system and method for an animal storage container that allows for rapid and sensitive control to vary the environmental conditions during a study (i.e., mixing rate within the animal cages is maximized). The advantage over having more rapid and sensitive control over the environmental conditions will allow for studies where better correlations can be drawn between the study variables and the physiological effects of sleep apnea. An additional advantage is more effective use of externally supplied gases, minimizing the cost of experimentation, and floor space needed (i.e., minimizing the laboratory footprint).

A third object of this invention is to provide a gas delivery system and method for an animal storage container that can accommodate the various animal cage designs already available, as discussed above, including standard, flow-through, and rotating/tether cages. The advantage of such a system and method is that it can be used with readily available materials. An additional advantage is the ability to design experiments at relatively lower costs.

A fourth object of this invention is to provide a gas delivery system and method for an animal storage container, with improved homogeneity, and where the gases (e.g., oxygen, carbon dioxide, ammonia), temperature, relative humidity, and pressure can be monitored closer to the animal's immediate environment than the commercial products currently available. The advantage of being able to make measurement closer to the animal is that results can be better correlated with the physiological effects of interest.

Additional objects of the invention are to provide a gas delivery system and method for an animal storage container that: 1) Contains a computer-based control and data acquisition interface for gas scheduling (e.g., oxygen scheduling), 2) eliminates extraneous stimuli to the animal (air jets, noise, etc.), and 3) can be adapted to address the design flaws inherent in the current state-of-the-art environmental chambers.

Other objects and advantages of the gas delivery system and method of this invention will be apparent to those skilled in the art in view of the detailed description of the invention set forth herein, and the non-exhaustive list of features, as follows.

It is a feature of an embodiment of this invention that the animal containers have one or more input gas locations.

It is an additional feature of an embodiment of this invention that the input gas locations comprise conduits (e.g., chimneys, inlets, ports, or hoses) containing open-cell foam or similar-acting material (filter paper, jet silencers, etc.) for acoustic damping, gas filtration, and flow diffusion.

It is an additional feature of an embodiment of this invention that animal cages or containers have connection to gases, such as, but not limited to, oxygen, nitrogen, and air. The gases can be supplied by tank, a compressor/reservoir system, or other commonly available supply source. The gases are optionally dried prior to delivery into the animal container or cage.

It is an additional feature of an embodiment of this invention that internal circulation (e.g., via axial or radial fans) is used to mix the animal container environment rapidly, while preventing (or diffusing) high velocity, directed Jets of supply gas from disrupting the internal contents of the container. The effectiveness of the fan(s) can be manipulated by appropriately positioning the fan(s), and adjusting how the fans are directed. The fans can be incorporated within the headspace or hollow area of the lid itself (e.g., when the lid is a composite lid or has separation between the top and bottom surfaces), or can be integral to the bottom or top surface of the lid.

It is an additional feature of an embodiment of this invention that single or multiple exhaust gas locations can be sized to allow for appropriate exhaust flow rates from the container or cage, thereby ensuring atmospheric pressure within the container in studies where this is important. This invention encompasses embodiments where the seal between the lid and the container bottom is relatively tight (and where there are exhaust ports incorporated into the lid or container), and other embodiments where the seal between the lid and container bottom is relatively loose, so that gas can exhaust through the relatively loose seal.

It is an additional feature of an embodiment of this invention that it uses a computer system containing provisions for an open-loop and/or closed-loop control algorithm; in certain embodiments, the algorithm can be activated by the animal's physiological metrics. The computer system may include a user interface to allow one to import and or prescribe, among other things: 1) a desired oxygen concentration schedule, 2) lighting conditions to simulate day and night, 3) data-logging specifics, 4) type of control desired, encompassing at least: proportional, proportional-integral, and proportional-integral-derivative modes of control, 5) capture of input signals through a data acquisition system, and, 6) output systems to control gas supply—e.g., output signals used to control valves, such as proportional or direct-acting solenoid valves, that vary the amounts of oxygen, nitrogen, and atmospheric air into the animal container.

It is an additional feature of an embodiment of this invention that it contains a gas analysis device (i.e., a sensor or detector) that samples the local environment within the container or cage at an appropriate sampling rate and phase lag. Oxygen, carbon dioxide, and/or ammonia detectors can be combined with pressure, temperature, and/or relative humidity sensors.

It is an additional feature of an embodiment of this invention that sensors, fans, and other sensitive devices be protected from animals, for example, by a metallic screen or grill that keeps the animals from damaging the devices.

It is an additional feature of an embodiment of this invention that lid dimensions can be adjustable to accommodate varying sizes of containers or cages.

It is additionally envisioned that embodiments of this invention can be useful in studying the effects of other gaseous, barometric, and thermal effects on laboratory animals, including, but not limited to: nitric oxide, cigarette smoke, carbon monoxide. This invention is not limited to sleep apnea-related studies.

It is an additional feature of this invention that it be adaptable to studies where the laboratory animals are either tethered or untethered.

### SUMMARY OF THE INVENTION

In one embodiment, the present invention is directed to a gas delivery system for an animal storage container, wherein the gas delivery system comprises:
a lid capable of engagement with an open first end of the container, wherein the lid has an interior face and an exterior face, and the lid comprises at least one conduit integral to the lid for delivering at least one gas from an external source to the container, wherein at least one of the conduits is at least partially filled with means for diffusing, filtering and acoustically damping the gas or gases passing through the conduit; and means for circulating gas within the container, wherein the means for circulating gas are integral to the lid.

In an alternate embodiment, the present invention is directed to a gas delivery system for an animal storage container, wherein the gas delivery system comprises:
a lid capable of engagement with an open first end of the container, wherein the lid has an interior face and an exterior face, and the lid comprises a first conduit integral to the lid for delivering at least one gas from an external source to the container and a second conduit integral to the lid for delivering at least one gas from an external source to the container, wherein the first and second conduits each are at least partially filled with open-cell foam and the first and second conduits are positioned in the lid to maximize gas mixing within the container; a circulating fan electrically connected to a power supply located external to the lid; at least one of the following detectors located within the container: an oxygen detector, a carbon dioxide detector, an ammonia detector, a gas pressure detector, a temperature detector, or a relative humidity detector; and means for achieving timed delivery of at least one gas to the container.

In another embodiment, the present invention is directed to a method of delivering gas to an animal storage container, wherein the method comprises:
providing a lid capable of engagement with an open first end of the container, wherein the lid has an interior face and an exterior face, and the lid comprises at least one conduit integral to the lid for delivering at least one gas from an external source to the container, wherein at least one of the conduits is at least partially filled with means for diffusing, filtering and acoustically damping the gas or gases passing through the conduit; and
providing means for circulating gas within the container, wherein the means for circulating gas are integral to the lid.

In another embodiment, the present invention is directed to a method of delivering gas to an animal storage container, wherein the method comprises:
providing a lid capable of engagement with an open first end of the container, wherein the lid has an interior face and an exterior face, and the lid comprises a first conduit integral to the lid for delivering at least one gas from an external source to the container and a second conduit integral to the lid for delivering at least one gas from an external source to the container, wherein the first and second conduits each are at least partially filled with open-cell foam;
providing a circulating fan electrically connected to a power supply located external to the lid;
at least one of the following detectors located within the container: an oxygen detector, a carbon dioxide detector, an ammonia detector, a gas pressure detector, a temperature detector, or a relative humidity detector; and
providing means for achieving timed delivery of at least one gas to the container.

In another embodiment, the present invention is directed to a gas delivery system for use in animal studies, wherein the gas delivery system comprises:
a containment and a lid capable of engagement with an open first end of a containment, wherein the lid further comprises a means for delivering at least one gas from an external source to the containment; means for circulating gas within the containment, wherein the means for circulating gas is integral to the lid; and means for allowing gas to exhaust from the containment.

### BRIEF DESCRIPTION OF THE DRAWINGS

FIG. 1 is a side view depicting an embodiment of this invention.

FIG. 2A is a side view depicting an additional embodiment of this invention.

FIG. 2B is a top view of the exhaust port depicted in FIG. 2A.

FIG. 3 is a graph of data collected demonstrating an embodiment of the present invention.

FIG. 4 depicts an unassembled view (FIG. 4A) and an assembled view (FIG. 4B) of an embodiment of this invention.

FIG. 5A depicts an exploded view of FIG. 5B, another embodiment of this invention.

FIG. 6 depicts another embodiment of this invention.

FIG. 7 depicts another embodiment of this invention.

FIG. 8 depicts another embodiment of this invention.

FIG. 9 depicts an exploded view of the embodiment depicted in FIG. 8.

### DETAILED DESCRIPTION OF THE INVENTION

This invention will be further understood in view of the following detailed description, and relative to the non-limiting embodiments provided herein, and with reference to the drawings.

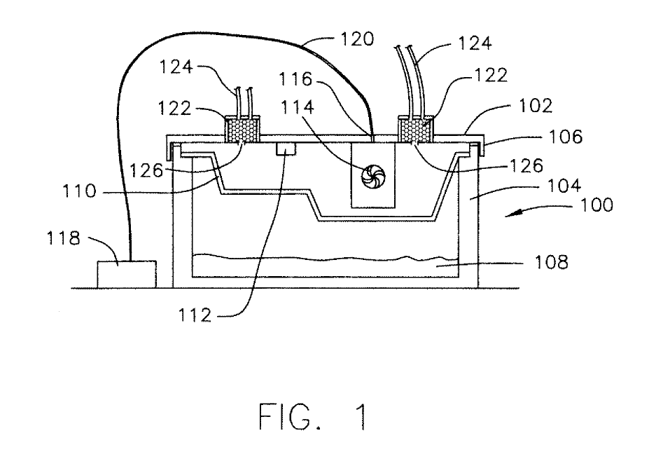

Referring now to FIG. 1, there is depicted a side view of one embodiment (100) of the invention. The animal storage system (100) of FIG. 1 comprises a lid (102) and a container (104). In a preferred embodiment, the lid (102) comprises a lip or edge (106) to keep the lid from sliding off of the container (104). The lid (102) can be made of any suitable material for use with an animal container. Preferred embodiments are made from plastics, such as acrylic. The container (104) can come from any standard, unmodified animal storage container, or can be custom made.

The animal storage container contains bedding (108) for the animals. In a preferred embodiment, the bedding also comprises the animal food mixed in. A grill (110) can be placed or hung in the container (104) to prevent the animals from chewing at the lid (102) or components capable of being contacted by the animals, such as a sensor (112), a circulating fan (114), or a water bottle (not shown), as well as other devices. Preferably, the grill (110) is made of metal.

In a preferred embodiment, the sensor (112) is an oxygen sensor. The sensor (112) can be placed anywhere throughout the container, but preferably above the grill (110), where it is protected from being damaged by the animals. Sensors are preferably attached to the lid (102), but can be placed anywhere within the container. Other sensors, besides oxygen sensors can be used (e.g., sensors that detect carbon dioxide, ammonia, temperature, pressure, and relative humidity, or other relevant parameters), and more than one sensor or detector (112) can be used at a time, depending on the animal study. All of these sensors are small enough to fit simultaneously above the grill (110). However, in alternate embodiments, tubing can be added to extract gas for analysis outside the chamber, and subsequent redelivery of the tested charge. This technique would decrease the response rate of the controller, but may be the only option for some detection systems.

Depending on the sensor and sophistication of the study, the sensor(s) (112) can be connected to, or communicate with, a control/recording system (not shown). Communication can be via remote signaling, or via a hard wire system (not shown). A hard wire connection can be achieved by feeding the connecting wire through a small hole or port, such as a port similar to port (116) shown in FIG. 1 for the power wire connected to the circulating fan (114). Additionally, the sensor (112) may be of the type that stores data for later downloading or access.

Placement of a sensor or detector (112) within the animal container, as shown in FIG. 1, enables monitoring of gas(es) as close as possible to the animal, closer than in any product to date, thereby ensuring fast response times and accuracy of gas concentration readings in the animal's immediate vicinity. Such placement also allows significant improvement in controllability of the environment, since the sensor readings can be linked to communicate with a control/recording system that can vary gas supply into the animal container based on sensor readings.

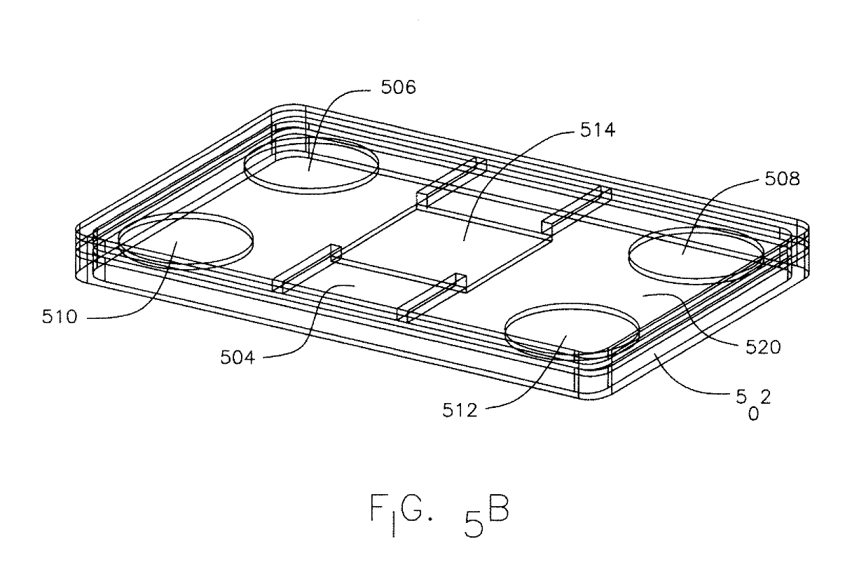

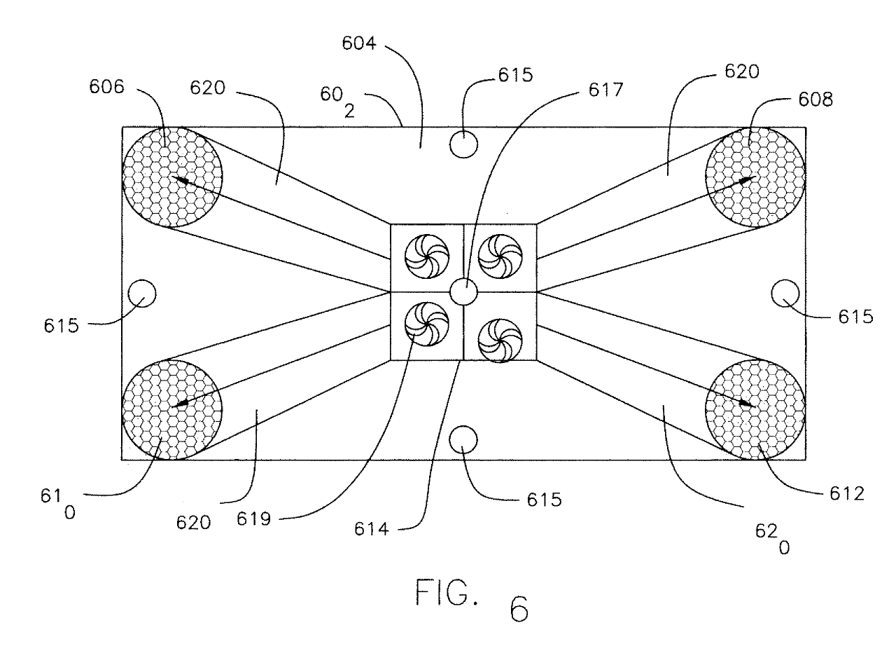

In a preferred embodiment, at least one circulation device, such as a fan (114), is used. The fan (114) is preferably attached to the underside of the lid (102), and above the grill (110). Fans can also be integral with the top of the lid, in close proximity to a conduit or other opening (e.g., small hole or port (116), noted below). The fan can be powered by an external power source (118) via wiring (120) that is fed through the small hole or port (116), about ⅛″ in diameter, in the lid (102). The wire can also be fed in through gaps between the lid (102) and the container (104). In alternate embodiments, the power source does not require an external connection, for example, if the fan (114) is powered by a battery or a self-contained power source, or power source integral to the lid. A programmable integrated circuit can also be attached to the lid, with wireless communication with valves and data acquisition. Any suitable fan, or other circulation device, can be used. For example, the fan can be an axial fan or a radial fan. The fan can also have multiple speeds, and can optionally communicate with a control system (not shown). Additional embodiments, discussed later herein, incorporate the fan within the lid (102) itself, where the lid has a headspace or gap between an upper surface and a lower surface (not shown, see, e.g., FIGS. 5 and 6). As will be described, such embodiments create currents and circulation from within the lid to the space within the container.

Studies were conducted with a system similar to the embodiment depicted in FIG. 1, having dimensions of 12 inches×7 inches×9 inches. In the studies, however, the circulation fan was omitted. An oxygen sensor was placed at various locations throughout the animal container. Results indicated that without the internal circulation fan, the oxygen concentration within the container could vary up to 1% absolute, depending on the location of the oxygen sensor. In other words, where the target oxygen concentration was set for 10%, actual concentrations at different locations within the container varied from about 9.5% to about 10.5%. Depending on the specific study, and the actual target gas concentrations of interest, such variation could limit the accuracy and types of conclusions that can be drawn in an animal study. These studies show that even within such a small environment of an animal container, as compared to the commercially available standard large containment chambers, an active mixing method is required within the animal container itself. While at least one circulation fan has been found to yield acceptable gas mixing and exchange, more fans may be preferable, depending on the study, container dimensions, and other factors.

Also depicted in FIG. 1 are two chimneys (122) as conduits for the supply gas(es). The chimney(s) (122) are connected to the supply gas(es) via hoses (124) on one end, and to the inside of the container via openings (126) in the lid (102) at the other end. The embodiment in FIG. 1 depicts four hoses. The number of hoses will vary, depending on the needs and design of the specific study, and the experimental layout. A lid (102) can have a single chimney (122), or may have more than one chimney (122); however, in other embodiments, discussed below, the conduit does not comprise a chimney, but rather other means for introducing gas into the container.

In a preferred embodiment, the lid (102) comprises more than one chimney located near opposite corners of the lid (102), promoting large-scale currents within the container so that gas circulation and homogeneity is maximized. The number and location of the chimney(s) (122) can be varied, preferably, in concert with varying the number, location, and types of fans (114), to achieve maximum circulation and homogeneity of the gas(es). An additional factor to consider when deciding where to locate gas supply is where animals tend to spend their time. It is preferable to avoid introducing the supply gas where the animals tend to sleep—for example, normally mice huddle together during sleeping hours under the water bottle.

The chimney(s) (122) are at least partially filled with material, such as open-cell foam or similarly-acting material (e.g., filter paper) for acoustic damping, gas filtration, and gas flow diffusion. Due to the diameter of a typical supply hose (124) and the volumetric flow rate of gas through the hoses during a typical study, delivery of gas into a study container would normally result in a high velocity gas jet or stream (e.g., ˜30 psi/˜200 kPa regulator gauge for a 45 liter per minute flow through a ¼ inch diameter hose) directed into the container or cage. Not only do these jets or streams of gas move the bedding and irritate the animals, the rapid flow is very loud. These velocity and acoustic stimuli may also affect the rodents in a way that confounds the data being sought in a study. Therefore, the chimneys (122) are filled with open-cell foam, or other material, that: 1) filter the inlet gas stream to remove foreign debris and water that may exist; 2) eliminate the noise from the rapid rush of gas through a relatively small orifice; and 3) diffuse the gas stream so that the high velocity jet slows down and flows through a much larger opening in the lid.

In a preferred embodiment, the engagement between the lid (102) and the container (104) forms a relatively loose seal, so that gas can exit or exhaust through the naturally existing gaps between the lid (102) and the container (104), in addition to any small holes (116) used for connecting the fan (114) or other devices to external components. These gaps and/or holes are small enough so that noticeable gas flow or movement between the inside and outside environments only occurs when a forced intake flow is present and/or when the fan is continuously turned on. The intake gas streams displace container gases outward; the fan pushes air out through the gaps. A sufficiently adequate seal may be desired to achieve low but predictable flow rates of gas through these gaps.

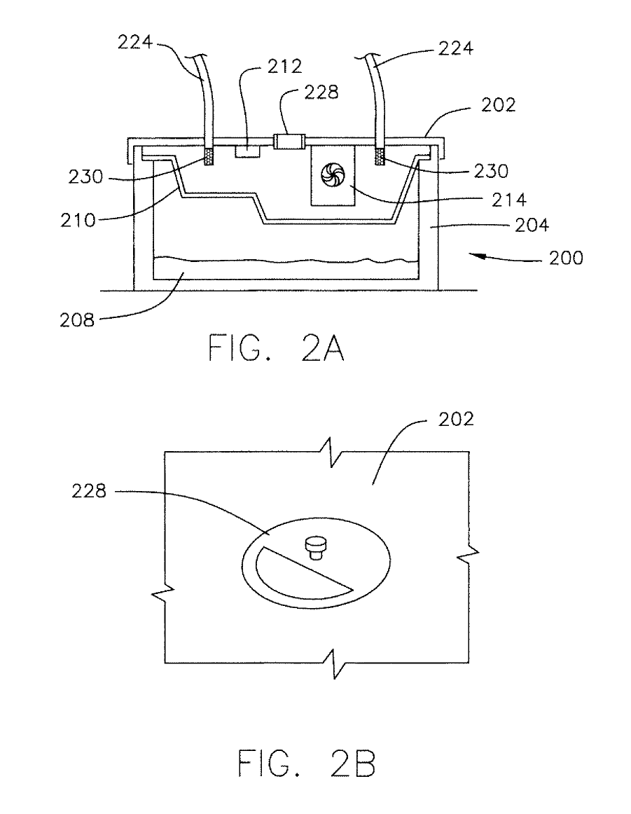

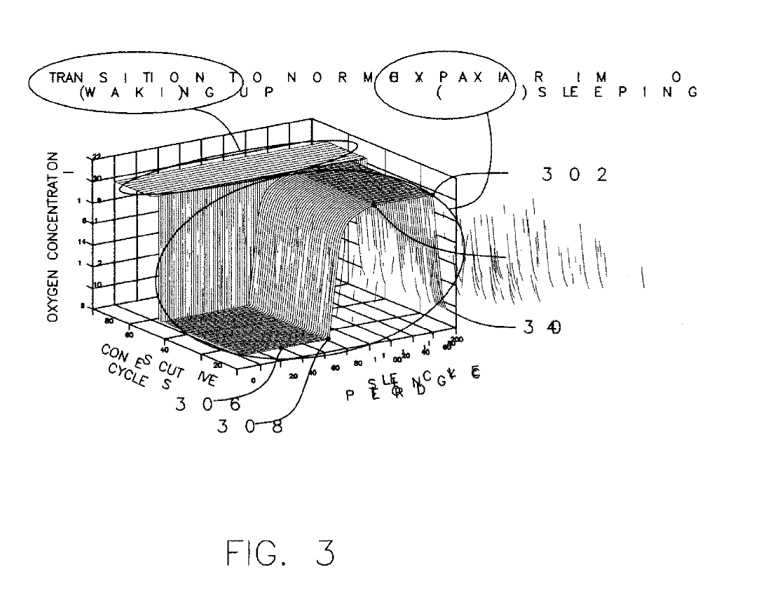

In an alternate embodiment, the engagement between the lid (102) and the container (104) forms a relatively tight fitting seal. In such an embodiment, it is preferable to have an additional opening in the lid, that can serve as a gas exhaust or exit port. FIG. 2A depicts the side view of an embodiment (200) that comprises an exhaust port (228). In a preferred embodiment, the exhaust port (228) is a variable opening exhaust port, where the size of the opening can be varied. A lid (202) may have more than one exhaust port (228). Also shown in FIG. 2A, for reference purposes, are: the lid (202), the container (204), the bedding (208), the grill (210), a fan (214), and a sensor or detecting device (212). Additionally shown are two hoses (224) that deliver the gas and serve as conduits. The hoses (224) feed through openings (226) in the lid (202), and contain silencing material (230) at the end of the hoses (224). In alternate embodiments, the silencing material can be located anywhere within the hose, or just external, but integral, to the hose. Also, note that in FIG. 2A the fan (214) does not require connection to an external power supply. FIG. 2B depicts a blown up top view of the exhaust port (228).

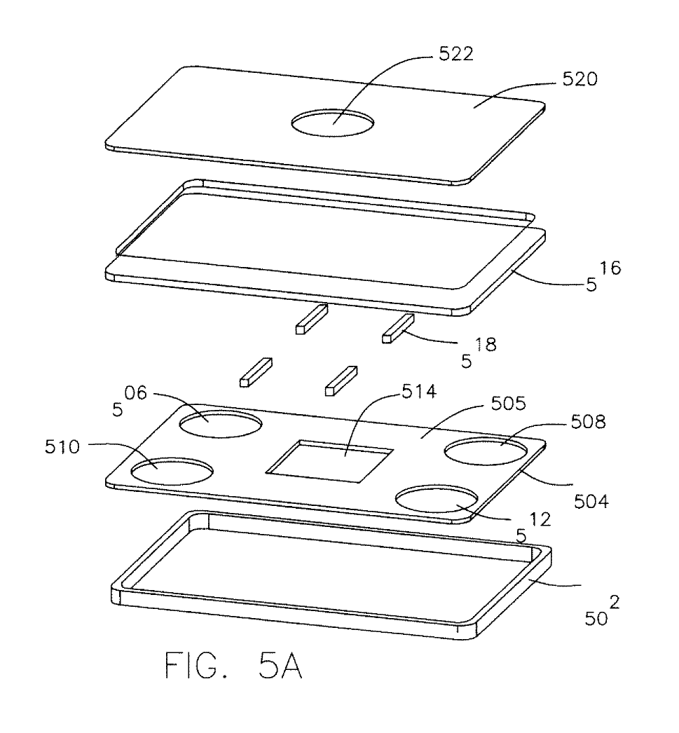

FIG. 4 depicts views of a lid of one embodiment of this invention, similar to the embodiment of the lid of FIG. 1. FIG. 4A is an exploded view of the lid, which comprises lid edge (402) which is in engagement with lid surface (404) which has an interior face (not shown) and exterior face (406). Lid surface (404) comprises openings (408), (410), and (424). Conduits (412) and (414) are located above openings (408) and (410), respectively. Cover plate (416) covers conduit (412) and cover plate (418) covers conduit (414). Cover plate (416) comprises openings (420) and cover plate (418) comprises openings (422). Conduits (412, 414) are at least partially filled with material (not shown) for diffusing, filtering and acoustically damping the gas or gases passing through the conduits (412, 414) via openings (420) and (422) respectively. Opening (424) is an opening, about ⅛ inches in diameter, primarily for allowing power wires or communication wires to pass, but also to provide a small opening for gas exchange. FIG. 4B depicts the assembled embodiment of FIG. 4A.

FIGS. 5A and 5B depict views of a lid of another embodiment of this invention. FIG. 5A is an exploded view showing a first lid frame (502) which surrounds a first lid surface (504) having an interior face (not shown) and an outer face (505) and openings (506, 508, 510, 512, 514). A second lid frame (516) rests on the outer edges of the outer face (505) of first lid surface (504), and a second lid surface (520) is suspended by optional spacers (518) and a second lid frame (516) above first lid surface (504), thereby providing an open area or head space between first lid surface (504) and second lid surface (520), which may have at least one opening therein (522). FIG. 5B depicts the assembled embodiment of FIG. 5A. In operation, first lid frame (502) is in engagement with an animal storage container (not shown). Opening (514) contains a plurality of gas circulating fans (not shown). Gases are supplied through the openings (FIG. 5A, (522)) in second lid surface (FIG. 5A, (520)). Circulation is achieved by fans that simultaneously draw air upward from the container, and gases downward from the supply source through the opening (FIG. 5A, (522)), thereby mixing the air and gases in the head space within the lid itself, and directing the mixture towards openings (506, 508, 510, 512) for delivery into the container. In a preferred embodiment, the fans are radial flow fans that draw gases from top and bottom and expel laterally (i.e., to the left and to the right). First lid surface (504) and second lid surface (520) may also contain additional openings (not shown) for providing gas passageways into and out of the container. The container itself may hold a plurality of individual cages or containers (not shown). This approach can be used to modify commercially available lids.

FIG. 6 depicts a bottom view of an additional embodiment. It is similar to the embodiment depicted in FIGS. 5A and SB, except that it has four additional openings (615, described below) in the first lid surface (604) and second lid surface (620, not shown). In FIG. 6, first lid frame (602) surrounds and supports first lid surface (604), which comprises openings (606, 608, 610, 612, 614). Openings (615, 617) are located in both the first lid surface (604) and second lid surface (620) (not shown). Preferably, opening (617) is the gas inlet for introducing gases such as air, nitrogen or oxygen into the container. Openings (615) are employed as gas exits to remove air and gas from the container (not shown) to the atmosphere—gases can exhaust through the openings (615) in the first lid surface (604), and continue exiting by flowing through overlaying openings (615) in the second lid surface (620, not shown). Openings (615) are preferably located in positions of minimum gas circulation in the container. Opening (617) is employed to draw air or gas into the head space (not shown). Gas circulating fans (619) are located in opening (614) as shown. Ducts (620) provide a mixture of entering gases and existing gases to conduits (606, 608, 610, 612) which communicate with the respective chambers (not shown) within the container.

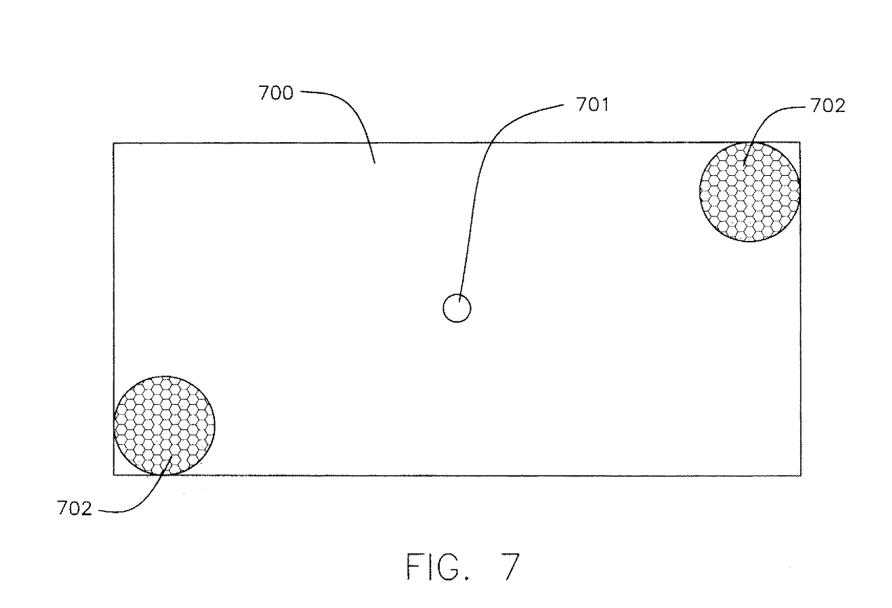

Another embodiment of the invention is depicted in FIG. 7. In FIG. 7, a top view of the lid (700) of this embodiment is shown. The lid can be a relatively flat lid, similar to the lid depicted in FIG. 1, or it can be a composite lid having headspace between lid surfaces, as depicted in FIG. 5. In this embodiment, a gas entry port (701) is located as shown. Gases are drawn into the container, through entry port (701) via a circulating fan (not shown) located proximate to the gas entry port (701). The drawn-in gases then mix with gases that already exist within the container, and force some exhaust through the exits (702), which are at least partially filled, or lined, with a material such as foam, filter paper, or other material. The filler/lining material provides some resistance to flow, or back pressure, which results in better control of the flow of the exiting gases. In one preferred embodiment, the circulating fan (not shown) is affixed to the inner surface of the lid below the gas entry port; in such an embodiment, the circulating fan is protected from the animal in the container by a cage or metallic grill (not shown) which separates the animal from the fan.

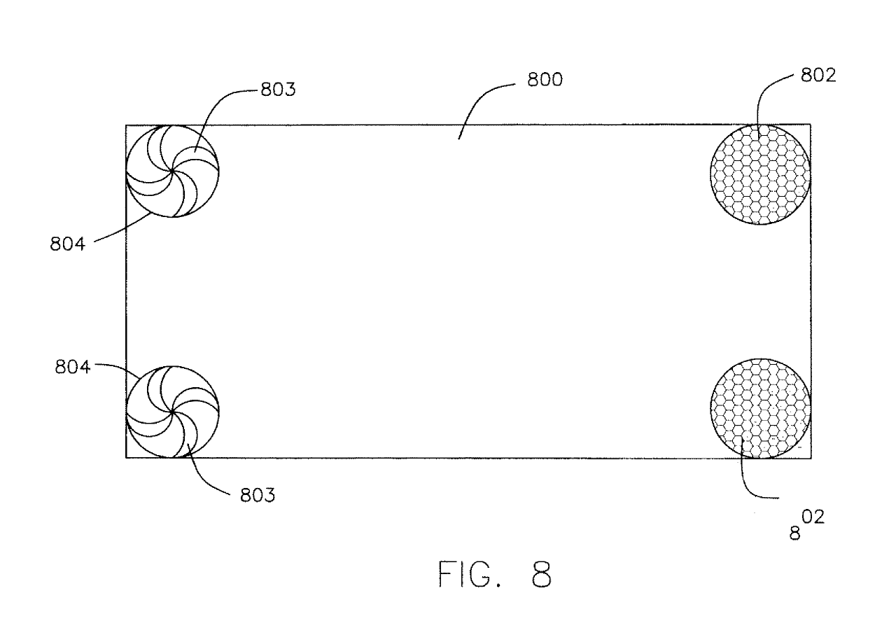

FIG. 8, depicts a top view of another embodiment (800). This embodiment is particularly useful for delivering gases to a plurality of animal storage containers located within an outer containment chamber—embodiment (800) can be used as a lid for the animal container(s) that reside within the outer containment. In FIG. 8, the gas entry ports (802) shown are at least partially filled with material such as foam for diffusing, filtering and acoustically damping the gas or gases passing through the gas entry ports prior to entry into the containers. Axial fans (803) located at exit ports (804) draw gas from the entry ports (802) into the containers located within the housing. A circulation fan (not shown) may also be located on the inner surface of the lid to assist in gas mixing within the housing and containers.

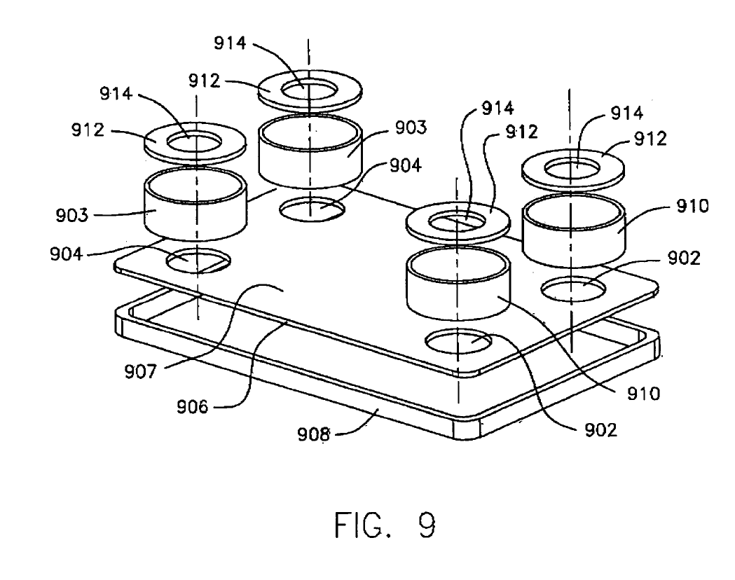

FIG. 9 depicts an exploded view of the embodiment depicted in FIG. 8. In FIG. 9, gas circulating axial flow fans (not shown) are located within conduits (903) located above openings (904) located in lid face (906) which has an exterior face (907) and an interior face (not shown). Lid face (906) is in engagement with lid edge (908). Gas entry ports or openings (902) are also located in lid face (906). Conduits (910) are located above openings (902). Cover plates (912) having opening (914) are in engagement with conduits (903, 910).

A useful feature of the present invention is that it can provide delivery of different gases to the animal container. Delivery can be controlled based on different criteria. For example, gas delivery can be set to follow a pre-determined schedule, or gas delivery can be based on feedback from gas sensors placed within the container. Control can be achieved by either an open-loop or a closed-loop system. In both open- and closed-loop systems, the gas flow is controlled and or monitored by a system of regulators, flowmeters, and valves, such as solenoid valves that respond to signals (e.g., from a timer, a computer control system, or manual inputs from an operator). Any regulator, flowmeter, and valve system suitable for control of gases may be used.

An open-loop control system is one in which the system is not continuously adjusted based on comparing a specific system response (e.g., gas concentration in the environment) with a desired standard for that quantity (called a setpoint), so that a minimum difference between the setpoint and the actual quantity is maintained. Open-loop protocols are set at the beginning of a study, and the only way to change the protocol is if there is periodic monitoring and intervention by an operator. For example, a sleep apnea study protocol may require cycles of rapid and intermittent changes in gas concentrations in an animal container. Open-loop systems are most useful where the system dynamics may be very simple, well-known, or disturbances in the system may not exist. Experimental setups (e.g., animal containers) slow to reach a target setpoint may require frequent monitoring and intervention, and lack in experimental precision. An advantage of the embodiments of the present invention, as discussed below, is that they respond rapidly and precisely to intermittent changes, and are therefore amenable to open-loop control without the need for operator monitoring and intervention, or even the electronic requirements of closed-loop counterparts.

In an open-loop system, control over a study protocol (e.g., the scheduling of the gas flows in a sleep apnea study) can be achieved manually, or using a device, such as a timer or computer. For example, the timed delivery of the gas(es) in a given study can be controlled via commonly used devices, such as electrically controlled valves (e.g., direct-acting solenoid valves) that are electronically actuated by a programmed timer, such as a ChronTrol® timer (the ChronTrol® timer contains four 120 VAC outlets that can be attached to and control electronic solenoid valves). In such an embodiment, the user programs the timer for the desired specific gas schedule (e.g., oxygen, nitrogen, atmospheric air, etc.). While preferred embodiments of this invention make use of direct-acting solenoid valves and the ChronTrol®, other control systems known in the art can also be used.

In another open-loop embodiment of this invention, the timing is programmed directly by the user via a computer using a graphical interface (i.e., the computer is used strictly as a timer, and not in a closed-loop manner, defined below). A graphical user interface provides a convenient and intuitive environment for setting up experimental conditions, data-logging and observation. For example, an interface may allow the user to set and modify parameters related to the hypoxia cycle, such as: duration, cycle profile, the degree of hypoxia in a single cycle, the degree of hyperoxia (if desired) in a single cycle, control of the fans and lighting, ramping of valve openings, the timing and length of hypoxia and normoxia cycles, sensor calibration, data-logging, data analysis, etc.

In another embodiment of the present invention, control is achieved by a closed-loop system. A closed-loop control system exists when an instrument detects the level of a specific system quantity, this level is compared against a setpoint, and system input is varied successively to minimize the difference between the setpoint and the actual quantity. Closed-loop control is typically accomplished via a computer system. In a closed-loop system, the computer is used to set up the initial experimental conditions (via a computer interface), and more importantly, to integrate signal inputs from detectors to control experimental parameters (e.g., affect actuation of valves that control gas flow in response to signals from sensors that detect gas concentrations in an animal container). Having a closed-loop system would allow the study to be fully-automated and to alleviate the need for an operator or technician to periodically check the study and manually make readjustments. As noted above, an experimental set-up that responds predictably to a schedule of intermittent changes may not need a closed-loop system.

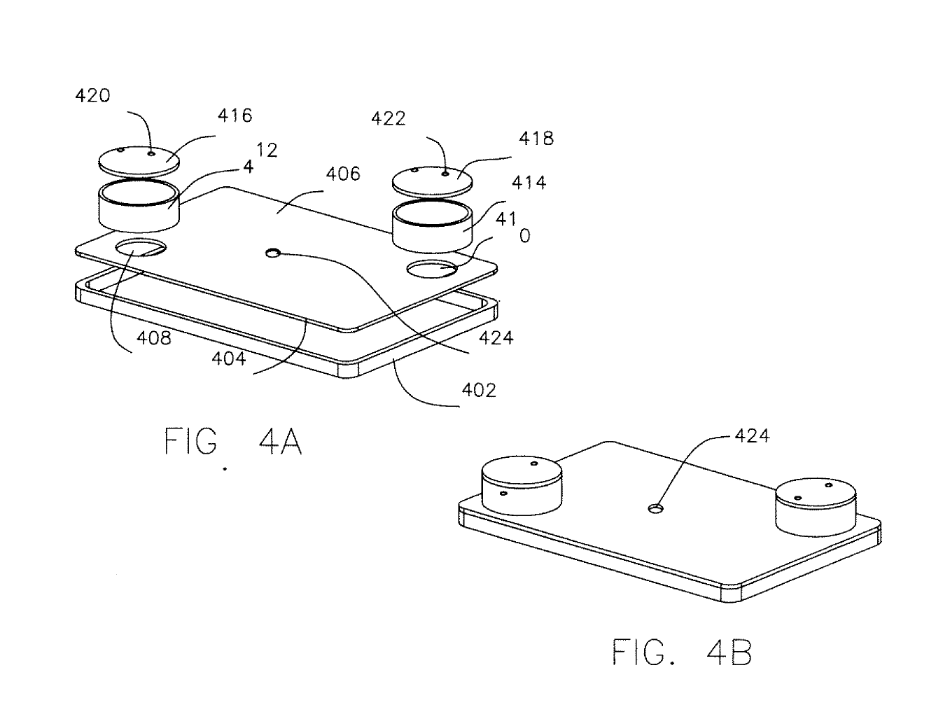

The invention may be illustrated by the following example, which is merely illustrative, and not meant to limit the present invention in any way. A study was conducted to demonstrate the effectiveness of an embodiment of this invention similar the one depicted in FIG. 1, with the following components: two chimneys; four hoses (two hoses for oxygen and two hoses for nitrogen, each pair originating from a single solenoid valve outlet via a ‘T’ fitting); one 12-VDC computer fan (from RadioShack®) (attached to the lid bottom, as per FIG. 1); one oxygen sensor (attached to the lid, as per FIG. 1), one additional hole (as in FIG. 1, (116)); and one ChronTrol® timer. The study involved a protocol of consecutive 180 second-long cycles repeated over 8 hours (which occurred within a broader study, having other phases as well—e.g., the “Transition to Normoxia” phase), and is tracked in the “Hypoxia Regime (Sleeping)” portion of the graph depicted in FIG. 3. Each cycle included approximately 90 seconds at hypoxia (see generally, FIGS. 3, (306)) and 90 seconds at normoxia (see generally FIG. 3, (310)). The protocol consisted of a set schedule where oxygen and nitrogen flows were controlled, and where the fan was turned off and on. Oxygen concentrations were monitored to determine how responsively the oxygen concentration varied with the protocol over the course of the 8 hour study. Protocol details were as follows:
1) At the beginning of each hypoxia cycle (see FIG. 3, (302)), the fans were on and the nitrogen valve was opened to allow flow at 45 liters per minute (LPM)—to be directed into two separate streams into the chimneys. Due to the entry of nitrogen into the animal container, the oxygen sensor detected a rapid decrease of oxygen, as depicted in the rapid decrease in oxygen concentration between points (302) and (304) of FIG. 3. (Note that FIG. 3 depicts results for 8 hours of consecutive cycles in the portion labeled “Hypoxia Regime.” The end of one cycle coincides with the beginning of the next cycle. For purposes of clarity, the data have been presented where point (302) depicts both, the end of one 180 second cycle, and the beginning of the subsequent 180 second cycle.).
2) After 5 seconds, the fan was turned off, and the nitrogen valve was deactivated (i.e, nitrogen flow was stopped). As shown in the flat portion of the graph, (306), the. oxygen concentration in the container reached and maintained a steady state level relatively quickly.
3) After 90 seconds (from the beginning of the nitrogen valve actuation; see FIG. 3 at (308)), the fan was turned on and the oxygen valve was opened, allowing oxygen to flow at a rate of about 7 LPM for five seconds only. The fan facilitated active mixing of the oxygen into the hypoxic environment, so that the system could rapidly return to normoxia (point (310) in FIG. 3 depicts that approximate steady state point of the return to normoxia). Additionally, unlike in steps 1 and 2 above, here, the fan was kept on to draw ambient room air into the container and to flush out the existing container air (i.e., promote large-scale leakage), assuring that the container environment contained normal atmospheric oxygen levels before the next hypoxia cycle.
4) At the end of the 180 second cycle (which coincides with the end of the 90-second return to normoxia) the nitrogen valves were turned on for 5 seconds to repeat steps 1 and 2 above (see FIG. 3, (302)) (note that the fan is already on from the preceding step, and is eventually turned off when the nitrogen valves were turned off).
5) The portion of FIG. 3 labeled ‘Transition to Normoxia’ denotes the period when the animal wakes up and no longer experiences simulated obstructive sleep apnea. This phase occurs outside of the 8-hour, 180-second cycles described above. During this next 16 hour phase, atmospheric air was being delivered to the container with the same scheduling, flow rates, and fan actuation as the hypoxia period, except that air from a compressor system was supplied to the animal cage in lieu of oxygen and nitrogen, at flow rates and timing similar to the 180-second cycle described above. The control of the air was performed through an electronic solenoid valve connected to the ChronTrol® timer.

As shown in FIG. 3, and as generally pointed out above, oxygen concentrations (vertical axis) varied from between about 20.9% and about 9.5% during the course of a cycle (right axis depicts the course of a single 180 second cycle). The transitions in oxygen concentration occurred relatively rapidly in response to the protocol, as reflected by the quick transition to steady state conditions (see e.g, FIGS. 3 at (302) to (304), and (308) to (310)). Additionally, results for consecutive cycles (see FIG. 3, left axis) overlay almost identically. Therefore, not only did the system respond rapidly to programmed changes during the 180 second cycle, but this rapid response was reliably repeated, cycle after cycle, over the 8 hour period studies, indicating that good open-loop control could be achieved with this embodiment. The collection of these type of data in such close proximity to the animals being studied is advantageous, and has not been previously accomplished.

The protocol described above was for an “experimental” animal group, where oxygen and nitrogen flows were varied. In practice, “experimental” groups are usually studied alongside a “control” group, so that influences of fan noise, air motion, and other stimuli can be decoupled from the effects of interest—the physiological effects associated with hypoxia. That is, having a “control” group allows one to assess whether differences between animals in the “experimental” container to those in the “control” are actually attributable to the oxygen cycling, or due to other stimuli in their respective environments. Accordingly, along with the experimental protocol described above (for the “experimental” group), in this study, a protocol was performed on an identical animal container as a “control” group. The “control” container was set up similarly to the “experimental” group, except that the gas infused to this system was atmospheric air using a dedicated solenoid valve programmed through the ChronTrol® timer. One aspect of the present invention is that it allows for properly-designed “control” containers, which can be used and controlled in concert with the experimental cages.

In summary, the invention described herein provides an improved system and method for animal studies requiring gas delivery. The system and method may be used in conjunction with commercially available laboratory animal cages, chambers, or other environmental systems. The system and method find particular use for sleep apnea studies, but are useful for other animal studies as well.

It should he understood that various changes and modifications to the preferred embodiments herein will be apparent to those skilled in the art. Such changes and modifications can be made without departing from the spirit and scope of this invention and without diminishing its attendant advantages. It is therefore intended that such changes and modifications be covered by the appended claims.

The invention claimed is:

1. A gas delivery system for an animal storage container, wherein the gas delivery system comprises:
a lid capable of engagement with an open first end of the container, wherein the lid has an interior face and an exterior face, and the lid comprises at least one conduit integral to the lid for delivering at least one gas from an external source to the container, wherein at least one of the conduits is at least partially filled with means for diffusing, filtering and acoustically damping the gas or gases passing through the conduit; and means for circulating gas within the container, wherein the means for circulating gas are integral to the lid.

2. The gas delivery system of claim 1, in which the system additionally comprises means for detecting the concentration of at least one gas within the container.

3. The gas delivery system of claim 1, in which the system additionally comprises means for achieving timed delivery of at least one gas to the container.

4. The gas delivery system of claim 1, in which at least one conduit is at least partially filled with open-cell foam.

5. The gas delivery system of claim 1, in which the lid additionally comprises at least one opening therethrough.

6. The gas delivery system of claim 1, in which the gas delivery system comprises at least one circulating fan located on the interior side of the lid, wherein the circulating fan is electrically connected to a power supply located external to the lid.

7. The gas delivery system of claim 1, in which the gas delivery system comprises at least one circulating fan located on the interior side of the lid, wherein the circulating fan is electrically connected to a power supply integral to the lid.

8. The gas delivery system of claim 1, in which at least one of the following detectors is located within the container: an oxygen detector; a carbon dioxide detector; an ammonia detector; a gas pressure detector; a temperature detector; or a relative humidity detector.

9. The gas delivery system of claim 1, in which a programmed timer is electronically connected to at least one electronically actuated valve which is connected to a source of at least one gas.

10. The gas delivery system of claim 1, in which a computer comprising a timing program is electronically connected to at least one electronically actuated valve which is connected to a source of at least one gas.

11. The gas delivery system of claim 10, in which the computer comprises a graphical user interface.

12. The gas delivery system of claim 10, in which the computer comprises a data logging module and a data observation module.

13. The gas delivery system of claim 1, in which means for preventing contact between an animal residing in the container and the interior face of the lid are located between the inner face of the lid and the container.

14. The gas delivery system of claim 13, in which the means for preventing contact between an animal residing in the container and the interior face of the lid comprises at least one of the following: an oxygen detector; a carbon dioxide detector; an ammonia detector; a gas pressure detector; a temperature detector; or a relative humidity detector.

15. The gas delivery system of claim 13, in which the means for preventing contact between an animal residing in the container and the interior face of the lid comprises means for providing food and water to the animal.

16. A gas delivery system for an animal storage container, wherein the gas delivery system comprises:
a lid capable of engagement with an open first end of the container, wherein the lid has an interior face and an exterior face, and the lid comprises a first conduit integral to the lid for delivering at least one gas from an external source to the container and a second conduit integral to the lid for delivering at least one gas from an external source to the container, wherein the first and second conduits each are at least partially filled with open-cell foam;
a circulating fan electrically connected to a power supply located external to the lid by wires;
at least one of the following detectors located within the container: an oxygen detector, a carbon dioxide detector, an ammonia detector, a gas pressure detector, a temperature detector, or a relative humidity detector; and
means for achieving timed delivery of at least one gas to the container.

17. A method of delivering gas to an animal storage container, wherein the method comprises:
providing a lid capable of engagement with an open first end of the container, wherein the lid has an interior face and an exterior face, and the lid comprises at least one conduit integral to the lid for delivering at least one gas from an external source to the container, wherein at least one of the conduits is at least partially filled with means for diffusing, filtering and acoustically damping the gas or gases passing through the conduit; and
providing means for circulating gas within the container, wherein the means for circulating gas are integral to the lid.

18. A method of delivering gas to an animal storage container, wherein the method comprises:
providing a lid capable of engagement with an open first end of the container, wherein the lid has an interior face and an exterior face, and the lid comprises a first conduit integral to the lid for delivering at least one gas from an external source to the container and a second conduit integral to the lid for delivering at least one gas from an external source to the container, wherein the first and second conduits each are at least partially filled with open-cell foam;
providing a circulating fan electrically connected to a power supply located external to the lid;
at least one of the following detectors located within the container: an oxygen detector, a carbon dioxide detector, an ammonia detector, a gas pressure detector, a temperature detector, or a relative humidity detector; and
providing means for achieving timed delivery of at least one gas to the container.

19. A gas delivery system for use in animal studies, wherein the gas delivery system comprises:
a containment and a lid capable of engagement with an open first end of a containment, wherein the lid further comprises a means for delivering at least one gas from an external source to the containment; means for circulating gas within the containment, wherein the means for circulating gas is integral to the lid; and means for allowing gas to exhaust from the containment.

20. The gas delivery system of claim 19, in which the containment is an external housing and further comprises at least one internal animal container.

21. The gas delivery system of claim 19, in which the containment is an animal container.

22. The gas delivery system of claim 19, wherein the means for delivering at least one gas from an external source to the containment is a chimney, a hose equipped with a silencer, or an opening in the lid.
